(19) United States
(12) Patent Application Publication
(10) Pub. No.: US 2006/0057179 A1
(43) Pub. Date: Mar. 16, 2006

(54) Therapeutic polyesters and polyamides

(76) Inventor: Karen J Giroux, Highland Park, NJ (US)

Correspondence Address: MAYER, BROWN, ROWE & MAW LLP, P.O. BOX 2828, CHICAGO, IL, 60690-2828, US

(21) Appl. No.: 10/503,838

(22) PCT Filed: Feb. 6, 2003
(86) PCT No.: PCT/US03/03718

§ 371 Date: Aug. 9, 2005

Related U.S. Application Data
(60) Provisional application No. 60/354,310, filed on Feb. 7, 2002.

(51) Int. Cl. A61K 31/74; A61F 13/00

(52) U.S. Cl. 424/422; 424/78.27

(57) ABSTRACT

Polymers (i.e. polyesters, polyamides, and polythioesters or a mixture thereof) which degrade hydrolytically into biologically active compounds are provided. Methods of producing these polymers, intermediates useful for preparing these polymers, and methods of using these polymers to deliver biologically active compounds to a host are also provided. Medical implants based on the polymers of the invention are also provided.

### BACKGROUND OF THE INVENTION

Polyesters are used routinely by those skilled in the art in various drug delivery systems.

For example, U.S. Pat. No. 5,942,252 describes a microcapsule comprising as its biocompatible excipient a poly(lactide-co-glycolide), poly(lactide), poly(glycolide), copolyoxalate, polycaprolactone, poly(lactide-co-caprolactone), poly(esteramide), polyorthoester, polyp-hydroxybutyric) acid and/or polyanhydride for use in delivering antigens or vaccines into and through mucosally-associated lymphoid tissue.

WO 99/29885 describes a process for degrading poly(ester-amides) and poly(ester-urethanes) encapsulating chemicals, drugs, enzymes, microorganisms and seeds by introducing the polymer to an aqueous nutrient solution and inoculating the solution with a culture containing a selected bacteria.

WO 98/36013 describes aliphatic-aromatic dihydroxy compounds for use as controlled drug delivery systems.

WO 97/39738 describes preparation of microparticles of a sustained release ionic conjugate comprising a free carboxyl group containing biodegradable polymers and a free amino group-containing drug.

### SUMMARY OF THE INVENTION

Polyesters, polythioesters, and polyamides which degrade into useful biologically active compounds have now been developed. Accordingly, the invention provides a polymer of the invention which is polymer comprising a backbone, wherein the backbone comprises ester, thioester, or amide linkages, and wherein the backbone comprises one or more groups that will yield a biologically active compound upon hydrolysis of the polymer.

The invention also provides a pharmaceutical composition comprising a polymer of the invention and a pharmaceutically acceptable carrier.

The invention also provides a therapeutic method for treating a disease in an animal comprising administering to an animal in need of such therapy, an effective amount of a polymer of the invention.

The invention also provides a method of delivering a biologically active compound to a host comprising administering to the host a biocompatible and biodegradable polymer of the invention, which degrades into the biologically active compound.

The invention provides a polymer of the invention for use in medical therapy, as well as the use of a polymer of the invention for the manufacture of a medicament useful for the treatment of a disease in a mammal, such as a human.

The invention also provides processes and intermediates disclosed herein that are useful for preparing a polymer of the invention.

The invention also provides a polymer or composition including a biologically active compound (active agent) or drug molecule of the invention that can be formed into a medical implant or microparticle or applied or coated onto a medical implant or microparticle.

### BRIEF DESCRIPTION OF DRAWINGS

FIG. 1. Southern Research's continuous microencapsulation process whereby a drug, polymer and polymer solvent dispersion is added to an mechanically agitated water/surfactant mixture to form an emulsion of microdroplets which is then extracted with water to remove solvent and form hardened microcapsules or microspheres for collection by centrifugation, filtration or the like.

FIG. 2. Illustration of several hollow needle-type carriers 12 for use in the invention.

FIG. 3. Illustration of placement of pellets, “biobullets,” or seeds 10 of the invention inside the hollow cavity or chamber of a bioerodable needle-type carrier.

### DETAILED DESCRIPTION OF THE INVENTION

Definitions

The following definitions are used, unless otherwise described:

The article “a” and “an” as used herein refers to one or to more than one (i.e. at least one) of the grammatical object of the article. By way of example, “an element” means one element or more than one element.

Halo is fluoro, chloro, bromo, or iodo.

Alkyl, alkoxy, etc. denote both straight and branched groups; but reference to an individual radical such as “propyl” embraces only the straight chain radical, a branched chain isomer such as “isopropyl” being specifically referred to.

Aryl denotes a phenyl radical or an ortho-fused bicyclic carbocyclic radical having about nine to ten ring atoms in which at least one ring is aromatic.

Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₆)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term ester linkage means —OC(═O)— or —C(═O)O—; the term thioester linkage means —SC(═O)— or —C(═O)S—; and the term amide linkage means —N(O)C(═O)— or —C(═O)N(R)—, wherein each R is a suitable organic radical, such as, for example, hydrogen, (C₁-C₆)alkyl, (C₃-C₆)cycloalkyl, (C₃-C₆)cycloalkyl(C₁-C₆)alkyl, aryl, heteroaryl, aryl(C₁-C₆)alkyl, or heteroaryl(C₁-C₆)alkyl.

The term “amino acid,” comprises the residues of the natural amino acids (e.g. Ala, Arg, Asn, Asp, Cys, Glu, Gln, Gly, His, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutarnate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York John Wiley & sons, Inc., and references cited therein).

The term “host” includes animals and plants.

The term “peptide” describes a sequence of 2 to 35 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 20, or 5 to 15 amino acids. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right

Polymers of the Invention

The biocompatible, biodegradable polyesters, polythioesters, and polyamides of the invention are useful in a variety of applications where delivery of a biologically active compound is desired. Examples of such applications include, but are not limited to, medical, dental and cosmetic uses.

The polymers of the invention may be prepared in accordance with methods commonly employed in the field of synthetic polymers to produce a variety of useful products with valuable physical and chemical properties. The polymers can be readily processed into pastes or solvent cast to yield films, coatings, microspheres and fibers with different geometric shapes for design of various medical implants, and may also be processed by compression molding and extrusion.

Polyesters and polyamides prepared in accordance with the present invention have average molecular weights of about 1500 Daltons up to about 100,000 Daltons, calculated by Gel Permeation Chromatography (GPC) relative to narrow molecular weight polystyrene standards. Preferred polyesters and polyamides have average molecular weights of about 1500 Daltons, up to about 50,000 Daltons calculated by Gel Permeation Chromatography (GPC) relative to narrow molecular weight polystyrene standards. Preferred polyesters and polyamides have average molecular weights of about 1500 Daltons, up to about 35,000 Daltons.

Medical implant applications include the use of polyesters, polythioesters, or polyamides to form shaped articles such as vascular grafts and stents, bone plates, sutures, implantable sensors, implantable drug delivery devices, stents for tissue regeneration, and other articles that decompose into non-toxic components within a known time period.

Polymers of the present invention can also be incorporated into oral formulations and into products such as skin moisturizers, cleansers, pads, plasters, lotions, creams, gels, ointments, solutions, shampoos, tanning products and lipsticks for topical application.

Although the invention provides homopolymers that are prepared from suitably functionalized biologically active compounds, Applicant has discovered that the mechanical and hydrolytic properties of polymers comprising one or more biologically active compounds can be controlled by incorporating a linking group (L) into the polymer backbone.

Preferably, the polymers of the invention comprise backbones wherein biologically active compounds and linker groups are bonded together through ester linkages, thioester linkages, amide linkages, or a mixture thereof. Due to the presence of the ester, thioester, and/or amide linkages, the polymers can be hydrolyzed under physiological conditions to provide the biologically active compounds. Thus, the polymers of the invention can be particularly useful as a controlled release source for a biologically active compound, or as a medium for the localized delivery of a biologically active compound to a selected site. For example, the polymers of the invention can be used for the localized delivery of a therapeutic agent to a selected site within the body of a human patient (i.e. within or near a tumor), where the degradation of the polymer provides localized, controlled, release of the therapeutic agent.

Biologically Active Compounds

The term “biologically active compound” includes therapeutic agents that provide a therapeutically desirable effect when administered to an animal (e.g. a mammal, such as a human). Biologically active compounds that can be incorporated into the polymers of the invention possess at least two functional groups that can each be incorporated into an ester, thioester, or amide linkage of a polymer (as discussed in detail below), such that, upon hydrolysis of the polymer, the therapeutic agent is obtained. These groups can independently be a hydroxy group (—OH), a mercapto group (—SH), an amine group (—NHR), or a carboxylic acid (—COOK).

The biologically active compounds can also comprise other functional groups (including hydroxy groups, mercapto groups, amine groups, and carboxylic acids, as well as others) that can be used to modify the properties of the polymer (e.g. for branching, for cross lining, for appending other molecules (e.g. another biologically active compound) to the polymer, for changing the solubility of the polymer, or for effecting the biodistribution of the polymer). Lists of therapeutic agents can be found, for example, in: Physicians' Desk Reference, 55 ed., 2001, Medical Economics Company, Inc., Montvale, N.J.; USPN Dictionary of USAN and International Drug Names, 2000, The United States Pharmacopeial Convention, Inc., Rockville, Md.; and The Merck Index, 12 ed., 1996, Merck & Co., Inc., Whitehouse Station, N.J. One skilled in the art can readily select therapeutic agents that possess the necessary functional groups for incorporation into the polymers of the invention from these lists.

Therapeutic agents that can be incorporated into the polymers of the invention include suitably functionalized analgesics or general or local anesthetics, anti-convulsants, anti-diabetic agents, anti-fibrotic agents, anti-infectives, anti-bacterials, anti-fungals, anti-neoplastics, cardioprotective agents, cardiovascular agents, anti-thrombotics, central nervous system stimulants, cholinesterase inhibitors, contraceptives, deodorants, dopamine receptor agonists, erectile dysfunction agents, fertility agents, gastrointestinal agents, gout agents, hormones, immunomodulators, immunosuppressives, migraine agents, non-steriodal anti-inflammatory drugs (NSADs), motion sickness agents, muscle relaxants, nucleoside analogs, neurodegenerative agents (e.g, Parkinson's disease), obesity agents, ophthalmic agents, osteoporosis agents, parasympatholytics, parasympathommetics, anti-anesthetics, prostaglandins, psychotherapeutic agents, respiratory agents, sedatives, hypnotics, skin and mucous membrane agents, smoking sessation agents, sympatholytics, urinary tract agents, vaginal agents, and vasodilators (see Physicians' Desk Reference, 55 ed., 2001, Medical Economics Company, Inc., Montvale, N.J., pages 201-202).

Linking Group “L”

The nature of the linking group “L” in a polymer of the invention is not critical provided the polymer of the invention possesses acceptable mechanical properties and release kinetics for the selected therapeutic application. The linking group L is typically a divalent organic radical having a molecular weight of from about 25 daltons to about 400 daltons. More preferably, L has a molecular weight of from about 40 daltons to about 200 daltons.

The linking group L typically has a length of from about 5 angstroms to about 100 angstroms using standard bond lengths and angles. More preferably, the linking group L has a length of from about 10 angstroms to about 50 angstroms.

The linking group may be biologically inactive, or may itself possess biological activity. The linking group can also comprise other functional groups (including hydroxy groups, mercapto groups, amine groups, carboxylic acids, as well as others) that can be used to modify the properties of the polymer (e.g. for branching, for cross linking, for appending other molecules (e.g. another biologically active compound) to the polymer, for changing the solubility of the polymer, or for effecting the biodistribution of the polymer).

Specific and Preferred Values

Specific and preferred values listed herein for radicals, substituents, groups, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, see-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutyltho, pentylthio, or hexylthio; (C₂-C₆)alkanoyloxy can be acetoxy, propanoyloxy, butanoyloxy, isobutanoyloxy, pentanoyloxy, or bexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific biologically active compound that can be incorporated into the polymers of the invention is atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin, alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor, ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; butorphanol; nalbuphine. streptozocin; doxorubicin; daunorubicin; plicamycin; idarubicin; mitomycin C; pentostatin; mitoxantrone; cytarabine; fludarabine phosphate; floxuridine; cladribine; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′-sulfinyldianiline; 4-sulfanilamidosalicylic acid; acediasulfone; acetosulfone; amikacin; amphotericin B; ampicillin; apalcillin; apicycline; apramycin; arbekacin; aspoxicillin; azidaimfenicol; aztreonam; bacitracin; bambermycin(s); biapenem; brodirnoprim; butirosin; capreomycin; carbenicillin; carbomycin; carumonam; cefadroxil; cefamandole; cefatrizine; cefbuperazone; cefclidin; cefdinir, cefditoren; cefepime; cefetamet; cefixime; cefinenoxime; cefininox; cefodizime; cefonicid; cefoperazone; ceforanide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin, dirithromycin; doxycycline; enoxacin; enviomycin; epicillin; erythromycin; flomoxef; fortimicin(s); gentainicin(s); glucosulfone solasulfone; gramicidin S; gramicidin(s); grepafloxacin; guamecycline; hetacillin; isepamicin; josamycin; kanamycin(s); leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; parornomycin; pazufloxacin; penicillin N; pipacycline; pipemidic acid; polymyxin; primycin, quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; Tomudex® (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol; dicoumarol; ethyl biscoumacetate; ethylidene dicoumarol; iloprost; lamifiban; taprostene; tioclomarol; tirofiban; amiprilose; bucillamine; gusperimus; mycophenolic acid; procodazole; romurtide; sirolimus (rapamycin); tacrolimnus; butethamine; fenalcomine; hydroxytetracaine; naepaine; orthocaine; piridocaine; salicyl alcohol; 3-amino-4-hydroxybutyric acid; aceclofenac; aiminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; gentisic acid; glucamethacin; glycol salicylate; meclofenamic acid; mefenamic acid; mesalamine; niflumic acid; olsalazine; oxaceprol; S-adenosylmethionine; salicylic acid; salsalate; sulfasalazine; or tolfenamic acid.

A preferred biologically active compound suitable for incorporation into polyesters of the invention is morphine, dopamine, bialamicol, or tetracycline.

A preferred biologically active compound suitable for incorporation into polyamides of the present invention is phenamidine, acriflavine, chloroazodin, arsphenamine, amicarbilide or aminoquinuride.

Another preferred biologically active compound that can be incorporated into a polymer of the invention is oxymorphone, buprenorphine, butorphanol, or nalbuphine.

Another preferred biologically active compound that can be incorporated into a polymer of the invention is methotrexate, doxorubicin, or daunorubicin.

Another preferred biologically active compound that can be incorporated into a polymer of the invention is atorvastatin, enalapril, ranitidine, pravastatin, cyclosporin, famotidine, leuprolide, acyclovir, lamivudine, budesonide, albuterol, indinavir, metformin, alendronate, nizatidine, zidovudine, carboplatin, metoprolol, lisinpril, captopril, salmeterol, cilastatin, benazepril, cefaclor, fluvastatin, quinapril, gemcitabine or vincristine.

Another preferred biologically active compound that can be incorporated into a polymer of the invention is a nonsteroidal anti-inflammatory drug, for example, a nonsteroidal anti-inflammatory drug as described in U.S. patent application (Ser. No. 09/732,516, filed 7 Dec. 2000), 3-amino-4-hydroxybutyric acid, aceclofenac, alminoprofen, amfenac, bromfenac, bromosaligenin, bumadizon, carprofen, diclofenac, diflunisal, ditazol, enfenamic acid, etodolac, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine, tolfenamic acid and the like.

Anther preferred biologically active compound that can be incorporated into a polymer of the invention is an anti-bacterial, for example, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin and the like.

Anther preferred biologically active compound that can be incorporated into a polymer of the invention is an anti-fungal, for example, azaserine, candicidin(s), chlorphenesin, dermostatin(s), filipin, fungichromin, mepartricin, nystatin, oligomycin(s), perimycin A, tubercidin and the like.

Anther preferred biologically active compound that can be incorporated into a polymer of the invention is an anti-cancer (e.g., carcinomas, sarcomas, leukemias and cancers derived from cells of the nervous system), including anti-neoplastic, for example, 6-azauridine, 6-diazo-5-oxo-L-norleucine, 6-mercaptopurine, aclacinomycin(s), ancitabine, anthramycin, azacitadine, azaserine, bleomycin(s), capecitabine, carubicin, carzinophillin A, chlorozotocin, chromomycin(s), cladribine, cytarabine, daunorubicin, denopterin, docetaxel, doxifluridine, doxorubicin, edatrexate, eflornithine, elliptinium, enocitabine, epirubicin, etoposide, floxuridine, fludarabine, gemcitabine, idarubicin, mannomustine, melphalan, menogaril, methotrexate, mitobronitol, mitolactol, mitomycin C, mitoxantrone, mopidamol, mycophenolic acid, nogalamycin, olivomycin(s), paclitaxel, pentostatin, peplomycin, pirarubicin, piritrexim, plicamycin, podophyllinic acid 2-ethylhydrazine, prednimustine, procarbazine, pteropterin, puromycin, ranimustine, streptonigrin, streptozocin, teniposide, thiamiprine, thioguanine, Tomudex® (N-[[5-[[(1,4-Dihydro-2-methyl-4-oxo-6-quinazolinyl)methyl]methylamino]-2-thienyl]carbonyl]-L-glutamic acid), toptecan, trimetrexate, tubercidin, ubenimex, vinblastine, vindesine, vinorelbine, zorubicin and the like.

Anther preferred biologically active compound that can be incorporated into a polymer of the invention is an anti-thrombotic, for example, argatroban, coumetarol, dicoumarol, ethyl biscoumacetate, ethylidene dicoumarol, iloprost, lamifiban, taprostene, tioclomarol, tirofiban and the like.

Anther preferred biologically active compound that can be incorporated into a polymer of the invention is an immunosuppressive, for example, 6-mercaptopurine, amiprilose, bucillamine, gusperimus, mycophenolic acid, procodazole, romurtide, sirolimus (rapamycin), tacrolimus, ubenimex and the like.

Anther preferred biologically active compound that can be incorporated into a polymer of the invention is a general or local anesthetic, for example, butethamine, fenalcomine, hydroxytetracaine, naepaine, orthocaine, piridocaine, salicyl alcohol and the like.

A specific value for L is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalky, (C₁-C₆)alkanoyl, (C₁-C₆)alkanoyloxy, (C₁-C₆)alkoxycarbonyl, (C₁-C₆)alkylthio, azido, cyano, nitro, halo; hydroxy, oxo (═O), carboxy, aryl, aryloxy, heteroaryl, and heteroaryloxy.

Another specific value for L is a divalent, branched or unbranched, saturated or unsaturated, hydrocarbon chain, having from 1 to 25 carbon atoms, wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

Another specific value for L is an amino acid.

Another specific value for L is a peptide

Another specific value for L is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—).

A more specific value for L is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₁₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

Another more specific value for L is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—).

Another more specific value for L is a divalent, branched or unbranched, saturated or unsaturated, hydrocarbon chain, having from 3 to 15 carbon atoms.

Another more specific value for L is a divalent, branched or unbranched, hydrocarbon chain, having from 3 to 15 carbon atoms.

A preferred value for L is a divalent, branched or unbranched, hydrocarbon chain, having from 6 to 10 carbon atoms.

A more preferred value for L is a divalent hydrocarbon chain having 7, 8, or 9 carbon atoms.

A most preferred value for L is a divalent hydrocarbon chain having 8 carbon atoms.

A specific polymer of the invention comprises one or more units of formula
—R₁-A-L-A- (I)
wherein R₁ is group that will provide a biologically active compound upon hydrolysis of the polymer; each A is independently an amide linkage, a thioester linkage, or an ester linkage; and L is a linking group.

Another specific polymer of the invention is a polymer which comprises one or more units of formula (II) in the backbone:
—R₂-A-L-A-R₃-A-L-A- (II)
wherein: R₂ and R₃ are each independently a group that will yield a biologically active compound upon hydrolysis of the polymer; each A is independently an amide, thioester, or ester linkage; and each L is independently a linking group. Such a polymer, wherein R₂ and R₃ are groups that will yield differing biologically active compounds upon hydrolysis of the polymer, are particularly useful for the administration of a combination of two therapeutic agents to an animal.

A preferred group of polyesters and polyamides includes polymers that are comprised of compounds containing at least two free alcohol or phenol groups or two at least two free amine groups available for reactions which co-polymerize with carboxylic acid groups or bis(acyl) chlorides.

Formulations

The polymers of the invention can be formulated as pharmaceutical compositions and administered to a mammalian host, such as a human patient in a variety of forms adapted to the chosen route of administration, i.e., orally, rectally, or parenterally, by intravenous, intramuscular, intraperitoneal, intraspinal, intracranial, topical or subcutaneous routes. For some routes of administration, the polymer can conveniently be formulated as micronized particles.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations preferably contain at least 0.1% of polymer by weight. The percentage of the compositions and preparations may, of course, be varied and may conveniently be between about 2 to about 80% of the weight and preferably 2 to about 60% of a given unit dosage form. The amount of polymer in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The polymer may also be administered intravenously, intraspinally, intracranially, or intraperitoneally by infusion or injection. Solutions of the polymer can be prepared a suitable solvent such as an alcohol, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile solutions or dispersions or sterile powders comprising the polymer containing the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the polymer in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the present polymers can be applied in pure form. However, it will generally be desirable to administer them as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Dosages

Useful dosages of the polymers can be determined by comparing their in vitro activity, and in vivo activity of the therapeutic agent in animal models. Methods for the extrapolation of effective dosages in mice, and other animals, to humans are known to the art; for example, see U.S. Pat. No. 4,938,949. Additionally, useful dosages can be determined by measuring the rate of hydrolysis for a given polymer under various physiological conditions. The amount of a polymer required for use in treatment will vary not only with the particular polymer selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses, per day. The sub-dose itself may be further divided, e.g., into a number of discrete loosely spaced administrations.

Combination Therapies

The polymers of the invention are also useful for administering a combination of therapeutic agents to an animal. Such a combination therapy can be carried out in the following ways: 1) a second therapeutic agent can be dispersed within the polymer matrix of a polymer of the invention, and can be released upon degradation of the polymer; 2) a second therapeutic agent can be appended to a polymer of the invention (i.e. not in the backbone of the polymer) with bonds that hydrolyze to release the second therapeutic agent under physiological conditions; 3) the polymer of the invention can incorporate two therapeutic agents into the polymer backbone (e.g. a polymer comprising one or more units of formula (II)) or 4) two polymers of the invention, each with a different therapeutic agent can be administered together (or within a short period of time).

Thus, the invention also provides a pharmaceutical composition comprising a polymer of the invention and a second therapeutic agent that is dispersed within the polymer matrix of a polymer of the invention. The invention also provides a pharmaceutical composition comprising a polymer of the invention having a second therapeutic agent appended to the polymer (e.g. with bonds that will hydrolyze to release the second therapeutic agent under physiological conditions).

The polymers of the invention can also be administered in combination with other therapeutic agents that are effective to treat a given condition to provide a combination therapy. Thus, the invention also provides a method for treating a disease in a mammal comprising administering an effective amount of a combination of a polymer of the invention and another therapeutic agent. The invention also provides a pharmaceutical composition comprising a polymer of the invention, another therapeutic agent, and a pharmaceutically acceptable carrier.

Preferred drug combinations for incorporation into the polymers or the compositions of the invention include the following: amoxicillin/clavulanic acid; and imipenem/cilastatin.

Preparation of Polymers of the Invention

Processes for preparing polymers of the invention are provided as further embodiments of the invention and are illustrated by the following procedures in which the meanings of the generic radicals are as given above unless otherwise qualified.

For example, a polymer of the invention can be prepared, as illustrated in Scheme I, from a biologically active compound of formula (X₁—R₁—X₂) and a linker precursor of formula Z₁-L-Z₂, wherein X₁, X₂, Z₁, and Z₂ are selected from the values in the table below. 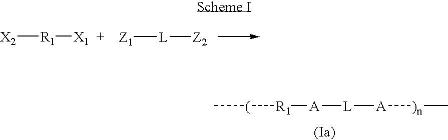
The biologically active compound and the linker precursor can be polymerized using well known synthetic techniques (e.g. by condensation) to provide a polymer of the invention (Ia) wherein each A is independently an ester linkage, a thioester linkage, or an amide linkage.

Depending on the reactive functional group (X₁ or X₂) of the biologically active compound, a corresponding functional group (Z₁ or Z₂) can be selected from the following table, to provide an ester linkage, thioester linkage, or amide linkage in the polymer backbone.

| Functional Group On | | |
|---|---|---|
| Biologically active | Functional Group On | |
| compound | Linker Precursor | Resulting Linkage In |
| (X₁ or X₂) | (Z₁ or Z₂) | Polymer |
| -COOH | -OH | Ester |
| -COOH | -NHR | Amide |
| -COOH | -SH | Thioester |
| -OH | -COOH | Ester |
| -SH | -COOH | Thioester |
| -NHR | -COOH | Amide |
| -SO₃H | -OH | Sulfate Ester |
| -OH | -SO₃H | Sulfate Ester |

As will be clear to one skilled in the art, suitable protecting groups can be used during the reaction illustrated in Scheme I (and in the reactions illustrated in Schemes II-XV below). For example, other functional groups present in the biologically active compound or the linker precursor can be protected during polymerization, and the protecting groups can subsequently be removed to provide the polymer of the invention. Suitable protecting groups and methods for their incorporation and removal are well known in the art (see for example Greene, T. W.; Wutz, P. G. M. “Protecting Groups In Organic Synthesis” second edition, 1991, New York, John Wiley & sons, Inc.).

Additionally, when a carboxylic acid is reacted with a hydroxy group, a mercapto group, or an amine group to provide an ester linkage, thioester linkage, or an amide linkage, the carboxylic acid can be activated prior to the reaction, for example, by formation of the corresponding acid, chloride. Numerous methods for activating carboxylic acids, and for preparing ester linkages, thioester linkages, and amide linkages, are known in the art (see for example Advanced Organic Chemistry: Reaction Mechanisms and Structure, 4 ed., Jerry March, John Wiley & Sons, pages 419-437 and 1281).

A polyester of the invention can be formed from a biologically active compound of formula (HO—R₁—OH) and from a linker precursor of formula HOOC-L-COOH as illustrated in Scheme II. 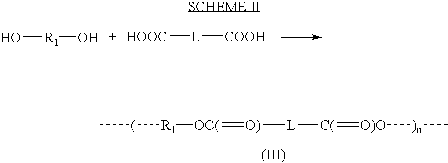
Reaction of the hydroxy groups of the biologically active compound with the carboxylic acids of the linker precursor provides a polymer of formula (III), which is a polymer of the invention.

A preferred biologically active dihydroxy compound that can be used to prepare a polyester of the invention is: amikacin; amphotericin B; apicycline; apramycin; arbekacin; azidamfenicol; bambermycin(s); butirosin, carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamicin(s); glucosulfone solasulfone; guamecycline; isepamicin; josamycin; kanamycin(s); leucomycin(s); lincomycin; lucensomycin; lymecycline; meclocycline; methacycline; micronomicin; midecamycin(s); minocycline; mupirocin; natamycin; neomycin; netilmicin; oleandomycin; oxytetracycline; paromomycin; pipacycline; podophyllinic acid 2-ethylhydrazine; primycin; ribostamycin; rifamide; rifampin; rifamycin SV; rifapentine; rifaximin, ristocetin; rokitamycin; rolitetracycline; rosaramycin; roxithromycin; sancycline; sisomicin; spectinomycin; spiramycin; streptomycin; teicoplanin; tetracycline; thiamphenicol; thiostrepton; tobramycin; trospectomycin; tuberactinomycin; vancomycin; candicidin(s); chlorphenesin; dermostatin(s); filipin; fungichromin; mepartricin; nystatin; oligomycin(s); perimycin A; tubercidin; 6-azauridine; aclacinomycin(s); ancitabine; anthramycin; azacitadine; bleomycin(s); carubicin; carzinophillin A; chlorozotocin; chromomycin(s); doxifluridine; enocitabine; epirubicin; gemcitabine; mannomustine; menogaril; atorvastatin; pravastatin; clarithromycin; leuprolide; paclitaxel; mitobronitol; mitolactol; mopidamol; nogalamycin; olivomycin(s); peplomycin; pirarubicin; prednimustine; puromycin; ranimustine; tubercidin; vindesine; zorubicin; coumetarol; dicoumarol; ethyl biscoumacetate; ethylidene dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuterol; indinavir; fluvastatin; streptozocin; doxorubicin; daunorubicin; plicamycin; idarubicin; pentostatin; mitoxantrone; cytarabine; fludarabine phosphate; floxuridine; cladribine; capecitabine; docetaxel; etoposide; topotecan; vinblastine; or teniposide.

A polyamide of the invention can be prepared using a procedure similar to that illustrated in Scheme II by replacing the biologically active dihydroxy compound in Scheme II with a suitable biologically active diamino compound. A preferred biologically active diamino compound that can be used to prepare a polymer of the invention is: 2-p-sulfanilyanilinoethanol; 4,4′-sulfinyldianiline; acediasulfone; acetosulfone; amikacin; apramycin; arbekacin; bacitracin; brodimorprim; butirosin; colistin; capreomycin; dapsone; dibekacin; enviomycin; gramicidin S; polymyxin; teicoplanin; fortimicin(s); gentamicin(s); glucosulfone solasulfone; grepafloxacin; imipenem; isepamicin; kanamycin(s); lymecycline; micronomicin; neomycin; netilmicin; p-sulfanilylbenzylamine; paromomycin; ribostamycin; ristocetin; sisomicin; sparfloxacin; spectinomycin; sulfachrysoidine; sulfamidochrysoidine; sulfoxone; tetroxoprim; thiazolsulfone; tobramycin; trimethoprim; edatrexate; eflornithine; mannomustine; mitoxantrone; peplomycin; piritrexim; procarbazine; pteropterin; trimetrexate; gusperimus; butethamine; naepaine; piridocaine; trospectomycin; tuberactinomycin; vancomycin; candicidin(s); mepartricin; perimycin A; ranitidine; famotidine; metformin; nizatidine; carboplatin; lisinopril; methotrexate; mitomycin bleomycin(s) or thioguanine.

A polythioester of the invention can be prepared using a procedure similar to that illustrated in Scheme II by replacing the biologically active dihydroxy compound in Scheme II with a suitable biologically active dimercapto compound.

A polysulfate ester of the invention can be formed by replacing the dicarboxylic acid linker compound with a disulfo acid compound A polyester/polyamide of the invention can be formed from a biologically active compound of formula (HRN—R₁—OH) and from a linker precursor of formula HOOC-L-COOH as illustrated in Scheme III. 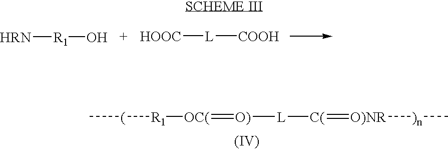
Reaction of the hydroxy group and the amino group of the biologically active compound with the carboxylic acids of the linker precursor provides a polymer of formula (M, which is a polymer of the invention.

A preferred biologically active hydroxy/amino compound that can be used to prepare a polyester/polyamide of the invention is: 2-p-sulfanilyanilinoethanol; 4-sulfanilamidosalicylic acid; amikacin; amphotericin B; apramycin; arbekacin; aspoxicillin; butirosin; capreomycin; cefadroxil; cefatrizine; cefdinir; cefprozil; dibekacin; dihydrostreptomycin; dirithromycin; enviomycin; gramicidin(s); teicoplanin; fortimicin(s); gentamicin(s); glucosulfone solasulfone; isepamicin; kanamycin(s); lucensomycin; lymecycline; meropenem; micronomicin; natamycin; neomycin; netilmicin; paromomycin; ribostamycin; ristocetin; sisomicin; spectinomycin; streptomycin; thiostrepton; tobramycin; trospectomycin; tuberactinomycin; vancomycin; candicidin(s); mepartricin; nystatin; perimycin A; tubercidin; anthramycin; azacitadine; bleomycin(s); carubicin; carzinophillin A; cytarabine; denopterin; elliptinium; epirubicin; gemcitabine; mannomustine; peplomycin; pirarubicin; pteropterin; puromycin; streptonigrin; tubercidin; ubenimex; vindesine; zorubicin; gusperimus; ubenimex; fenalcomine; hydroxytetracaine; orthocaine; 3-amino-4-hydroxybutyric acid; etofenamate; fepradinol; mesalamine; S-adenosylmethionine; leuprolide; acyclovir; paclitaxel; lamivudine; albuterol; indinavir; alendronate; zidovudine; metoprolol; amoxicillin; salmeterol; imipenem; doxorubicin; daunorubicin; idarubicin; pentostatin; mitoxantrone; fludarabine phosphate; floxuridine; cladribine; vinorelbine; vincristine; or vinblastine.

A polythioester/polyamide of the invention can be prepared using a procedure similar to that illustrated in Scheme II by replacing the hydroxy/amino biologically active compound in Scheme II with a suitable mercapto/amino biologically active compound.

A polyamide of the invention can be formed from a biologically active compound of formula (HOOC—R₁—COOH) and from a linker precursor of formula HRN-L-NRH as illustrated in Scheme IV. 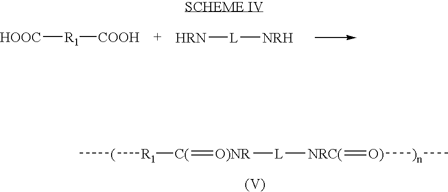
Reaction of the carboxylic acid groups of the biologically active compound with the amino groups of the linker precursor provides a polymer of formula (V), which is a polymer of the invention.

A preferred biologically active dicarboxylic acid compound that can be used to prepare a polyamide of the invention is: bambermycin(s); carbenicillin; carzinophillin A; cefixime; cefminox; cefpimizole; cefodizime; cefonicid; ceforanide; cefotetan; ceftibuten; cephalosporin C; denopterin; edatrexate; moxalactam; olsalazine; penicillin N; quinacillin; temocillin; ticarcillin; Tomudex® (N-[[5-[[(1,4-Dihydro-2-methyl-4-oxo-6-quinazolinyl)methyl]methylamino]-2-thienyl]carbonyl]-L-glutamic acid); lisinopril; cilastatin; ceftazidime; or methotrexate.

A polyester of the invention can be prepared using a procedure similar to that illustrated in Scheme IV by replacing the diamino linker precursor with a dihydroxy linker precursor. Similarly, a polyester/polyamide of the invention can be prepared using a procedure similar to that illustrated in Scheme IV by replacing the diamino linker precursor with an hydroxy/amino linker precursor; and a polythioester/polyamide of the invention can be prepared using a procedure similar to that illustrated in Scheme IV by replacing the diamino linker precursor with an mercapto/amino linker precursor.

A polyester of the invention can be formed from a biologically active compound of formula (HO—R₁—COOH) and from a linker precursor of formula HO-L-COOH as illustrated in Scheme V. 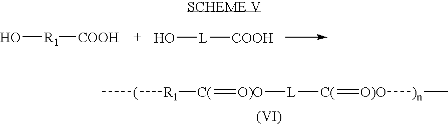
Reaction of the hydroxy group and the carboxylic acid of the biologically active compound, with the carboxylic acid and the hydroxy group of the linker precursor provides a polymer of formula (VI), which is a polymer of the invention.

A preferred biologically active hydroxy/carboxylic acid compound that can be used to prepare a polymer of the invention is: 4-sulfanilamidosalicylic acid; amphotericin B; apalcillin; apicycline; aspoxicillin; bambermycin(s); biapenem; cefadroxil; cefamandole; cefatrizine; cefbuperazone; cefdinir; cefonicid; cefoperazone; cefpiramide; cefprozil; enviomycin; teicoplanin; flomoxef; glycol salicylate; lucensomycin; lymecycline; meropenem; moxalactam; mupirocin; nadifloxacin; natamycin; panipenem; podophyllinic acid 2-ethylhydrazine; ritipenem; salazosulfadimidine; sulfaloxic acid; vancomycin; 3-amino-4-hydroxybutyric acid; candicidin(s); carzinophillin A; denopterin; diflunisal; fendosal; gentisic acid; iloprost; lamifiban; mesalamine; nystatin; olsalazine; oxaceprol; pteropterin; romurtide; salicylic acid; salsalate; streptonigrin; sulfasalazine; taprostene; ubenimex; amoxicillin; pravastatin; imipenem; mycophenolic acid; or fluvastatin.

A polyester/polyamide of the invention can be prepared using a procedure similar to that illustrated in Scheme V by replacing biologically active hydroxy/carboxylic compound with a biologically active amino/carboxylic acid compound. A preferred biologically active amino/carboxylic acid compound that can be used to prepare a polymer of the invention is: 3-amino-4-hydroxybutyric acid; 4-sulfanilamidosalicylic acid; 6-diazo-5-oxo-norleucine; aceclofenac; acediasulfone; alminoprofen; amfenac; amphotericin B; ampicillin; argatroban; aspoxicillin; azaserine; aztreonam; bromfenac; bumadizon; candicidin(s); carprofen; carumonam; carzinophillin A; cefadroxil; cefatrizine; cefclidin; cefdinir, cefditoren; cefepime; cefetamet; cefixime; cefinenoxime; cefininox; cefodizime; ceforanide; cefotaxime; cefotiam; cefozopran; cefpirome; cefprozil; cefroxadine; ceftazidime; cefteram; ceftibuten; ceftriaxone; cefuzonam; cephalexin; cephaloglycin; cephalosporin C; cephradine; clinafloxacin; cyclacillin; denopterin; edatrexate; eflornithine; enfenamic acid; enoxacin; epicillin; etodolac; enviomycin; teicoplanin; flufenamic acid; grepafloxacin, hetacillin; imipenem; lomefloxacin; lucensomycin; lymecycline; meclofenamic acid; mefenamic acid; meropenem; mesalamine; natamycin; niflumic acid; norfloxacin; nystatin; pazufloxacin; penicillin N; pipemidic acid; procodazole; pteropterin; S-adenosylmethionine; sparfloxacin; streptonigrin; succisulfone; sulfachrysoidine; temafloxacin; tigemonam; tirofiban; tolfenamic acid; tosufloxacin; trovafloxacin; ubenimex; vancomycin; enalapril; amoxicillin; ciprofloxacin; diclofenac; lisinopril; ceftriaxone; cilastatin; benazepril; cefaclor, ceftazidime; quinapril; melphalan; or methotrexate.

A polythioester/polyester of the invention can be prepared using a procedure similar to that illustrated in Scheme V by replacing biologically active hydroxy/carboxylic compound with a biologically active mercapto/carboxylic acid compound. A preferred biologically active mercapto/carboxylic acid compound that can be used to prepare a polymer of the invention is bucillamine or captopril.

A polysulfonamide of the invention can be prepared using a procedure similar that illustrated in Scheme V by replacing the biologically active hydroxy/carboxylic compound with a biologically active amine/sulfo acid compound. A preferred biologically active amine/sulfo acid compound that can be used to prepare a polymer of the invention is: sulfanilic acid or sulfoxone.

In the polymers of formulae (I, and III-VI) illustrated in Schemes I-V above, R₁, A, L, and R can have any of the values, specific values, or preferred values described herein.

The co-polymerization of morphine with a diacid chloride to provide a polyester of the invention is depicted in Scheme VI. 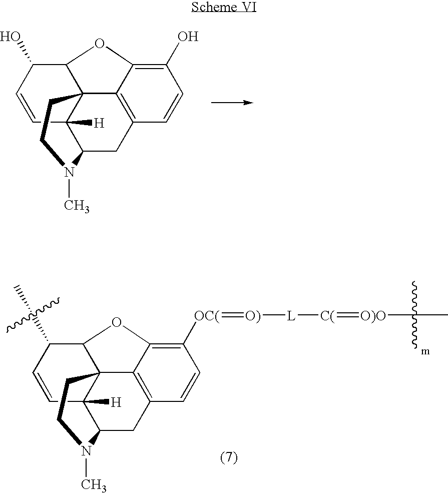
In the reaction illustrated in Scheme VI, the linking group L is preferably —(CH₂)ₓ—, and more preferably, L is —(CH₂)₈—. A polymer of formula (7) wherein L has any of the values, specific values, or preferred values described herein is a preferred polymer of the invention. For a polymer of formula (7), m is an integer that is greater than or equal to 2.

The co-polymerization of dopamine with bis(acyl) chloride to provide a polyester of the invention is depicted in Scheme VII. 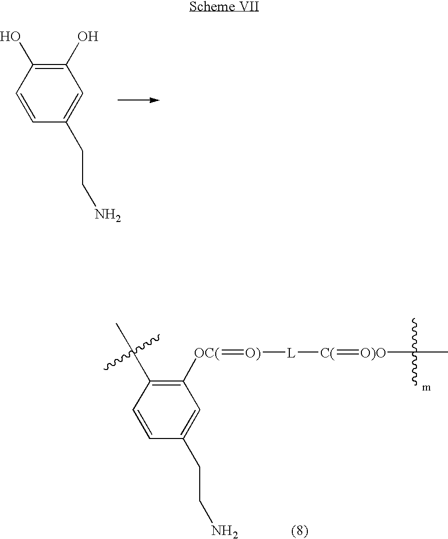
In the reaction illustrated in Scheme VII, the linking group L is preferably —(CH₂)ₓ—, and more preferably, L is —(CH₂)₈—. A polymer of formula (8) wherein L has any of the values, specific values, or preferred values described herein is a preferred polymer of the invention. For a polymer of formula (8), m is an integer that is greater than or equal to 2. Prior to the polymerization illustrated in Scheme VII, the amino group of dopamine can be protected with a suitable protecting group, which can subsequently be removed, to provide the polymer of the invention.

It should be noted that dopamine can also be incorporated into a polyester/polyamide of the invention by reacting the amino group and either hydroxy group of dopamine with a compound of formula HOOC-L-COOH, or an activated derivative thereof to provide a compound of formula (21) or (22): 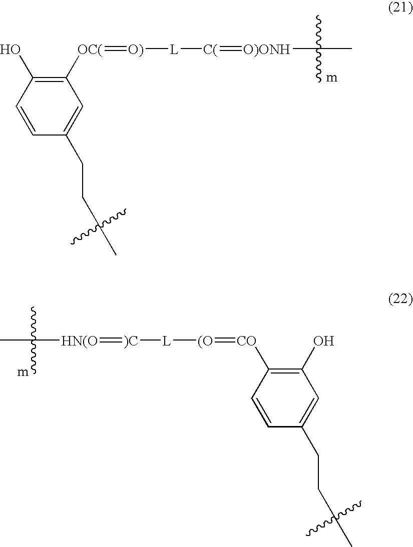
Prior to the polymerization, the hydroxy group which will not be polymerized can be protected with a suitable protecting group, which can subsequently be removed to provide the polymer of the invention.

The copolymerization of acriflavine to provide a polyamide of the invention is depicted in Scheme VIII. 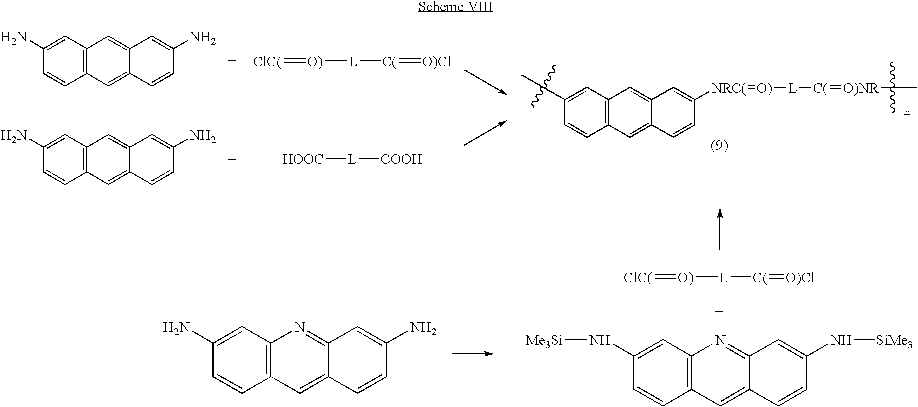
The diamino groups of acriflavine are copolymerized in solution preferably high-boiling point organic solvent such as dimethylformamide) with an activated dicarboxylic acid (e.g. sebacoyl chloride). The polyamide is isolated by methods well known in the art. Alternatively, the amino groups can be reacted with a dicarboxylic acid by employing high temperatures (e.g. in the melting range), or a coupling agent. This process of making polyamides is also well known to those skilled in the art. In yet another embodiment, the diamino groups can be activated in the presence of hexamethylsilazine to form silylated amines. The silylated amines can then be allowed to react with an activated dicarboxylic acid (e.g. sebacyl chloride) to provide a polymer of the invention.

In the reaction illustrated in Scheme VIII, the linking group L is preferably —(CH₂)ₓ—, and more preferably, L is —(CH₂)₈—. A polymer of formula (9) wherein L has any of the values, specific values, or preferred values described herein is a preferred polymer of the invention. For a polymer of formula (9), m is an integer that is greater than or equal to 2.

The preparation of a polymer of the invention comprising methotrexate is illustrated in Scheme IX.

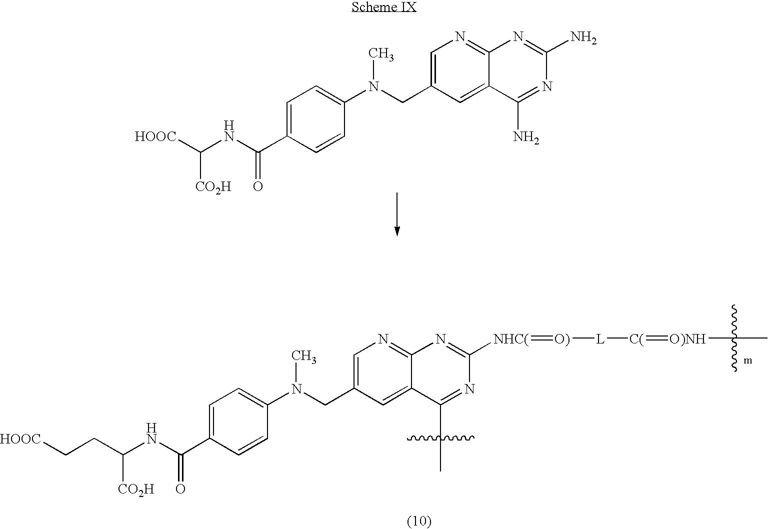

In the reaction illustrated in Scheme IX, the linking group L is preferably —(CH₂)ₓ—, and more preferably; L is —(CH₂)₈—. A polymer of formula (10) wherein L has any of the values, specific values, or preferred values described herein is a preferred polymer of the invention. For a polymer of formula (10), m is an integer that is greater than or equal to 2. Prior to the polymerization illustrated in Scheme IX, the carboxylic acids of methotrexate can be protected with suitable protecting groups, which can subsequently be removed, to provide the polymer of the invention.

It will be appreciated by one skilled in the art that a polymer of the invention of the following formula (20): 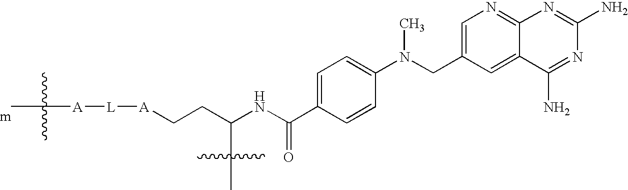
wherein each A is independently an ester linkage, a thioester linkage, or an amide linkage can be prepared as illustrated in Scheme X by selecting a linker precursor with the appropriate functionality. For a compound of formula (20) the linking group L is preferably —(CH₂)ₓ—, and more preferably, L is —(CH₂)₈—. A polymer of formula (20) wherein L and each A has any of the values, specific values, or preferred values described herein is a preferred polymer of the invention. For a polymer of formula (20), m is an integer that is greater than or equal to 2.

The preparation of another polymer of the invention comprising methotrexate is illustrated in Scheme X.

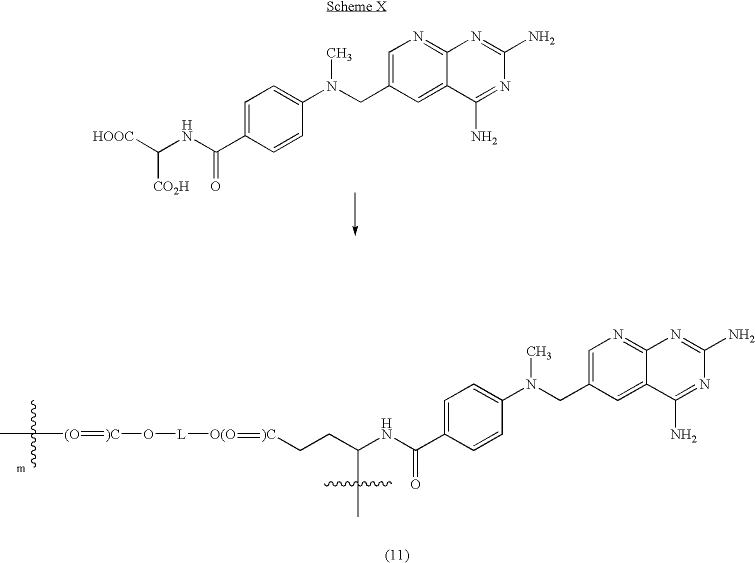

In the reaction illustrated in Scheme X, the linking group L is preferably —(CH₂)ₓ—, and more preferably, L is —(CH₂)₈—. A polymer of formula (11) wherein L has any of the values, specific values, or preferred values described herein is a preferred polymer of the invention. For a polymer of formula (11), m is an integer that is greater than or equal to 2. Prior to the polymerization illustrated in Scheme X, the amino groups of methotrexate can be protected with suitable protecting groups, which can subsequently be removed, to provide the polymer of the invention.

The preparation of another polymer of the invention comprising methotrexate is illustrated in Scheme XI.

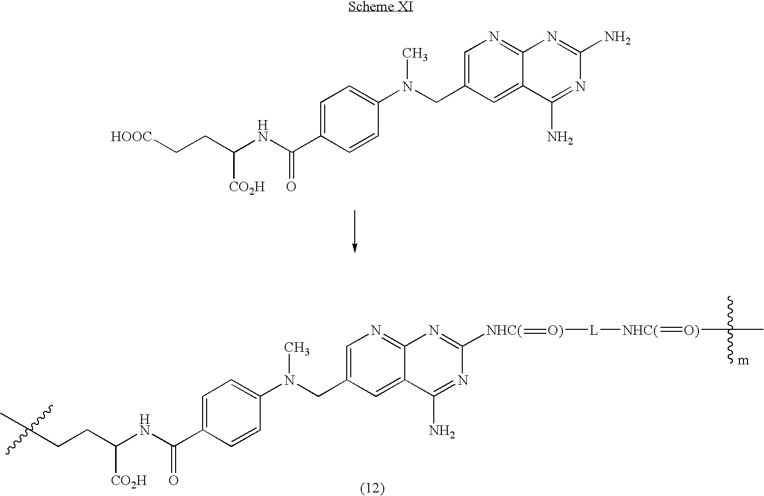

In the reaction illustrated in Scheme XI, the linking group L is preferably —(CH₂)ₓ—, and more preferably, L is —(CH₂)₈—. A polymer of formula (12) wherein L has any of the values, specific values, or preferred values described herein is a preferred polymer of the invention. For a polymer of formula (12), m is an integer that is greater than or equal to 2. Prior to the polymerization illustrated in Scheme XI, the carboxylic acid and amino group of methotrexate that are not reacted to form the polymer can be protected with suitable protecting groups, which can subsequently be removed, to provide the polymer of the invention.

A polymer of the invention that comprises methotrexate is particularly useful for treating psoriasis, inflammatory bowel disease, skin cancer, or brain tumors. Such a polymer is also particularly useful as an anti-neoplastic agent-anti-infective agent, and for local administration of an anti-tumor agent following a lumpectomy or mastectomy.

The preparation of a polymer of the invention comprising doxorubicin is illustrated in Scheme XII.

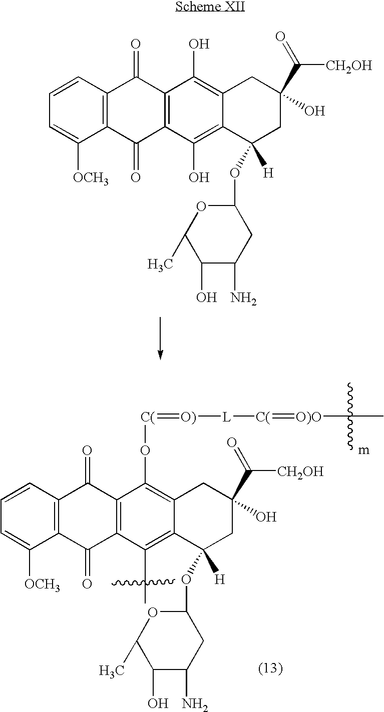

In the reaction illustrated in Scheme XII, the linking group L is preferably —(CH₂)ₓ—, and more preferably; L is —(CH₂)₈—. A polymer of formula (13) wherein L has any of the values, specific values, or preferred values described herein is a preferred polymer of the invention. For a polymer of formula (13), m is an integer that is greater than or equal to 2. Prior to the polymerization illustrated in Scheme XII, the functional groups of doxorubicin that are not reacted to form the polymer can be protected with suitable protecting groups, which can subsequently be removed, to provide the polymer of the invention.

A polymer of the invention that comprises doxorubicin is particularly useful for local administration as an anti-tumor (e.g. brain tumor) agent or anti-neoplastic agent.

The preparation of a polymer of the invention comprising daunorubicin is illustrated in Scheme XIII.

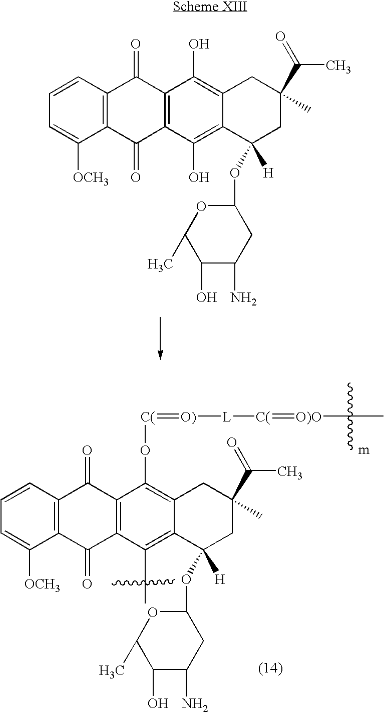

In the reaction illustrated in Scheme XIII, the linking group L is preferably —(CH₂)ₓ—, and more preferably, L is —(CH₂)₈—. A polymer of formula (14) wherein L has any of the values, specific values, or preferred values described herein is a preferred polymer of the invention. For a polymer of formula (14), m is an integer that is greater than or equal to 2. Prior to the polymerization illustrated in Scheme XIII, the functional groups of daunorubicin that are not reacted to form the polymer can be protected with suitable protecting groups, which can subsequently be removed, to provide the polymer of the invention.

A polymer of the invention that comprises daunorubicin is particularly useful for local administration as an anti-tumor (e.g. brain tumor) agent or an anti-neoplastic agent.

The preparation of a polymer of the invention comprising 5-aminosalicylic acid is illustrated in Scheme XIV. 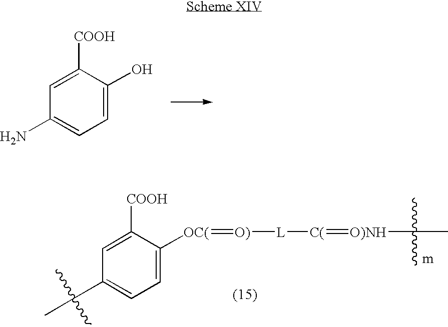
In the reaction illustrated in Scheme XIV, the linking group L is preferably —(CH₂)ₓ—, and more preferably, L is —(CH₂)₈—. A polymer of formula (15) wherein L has any of the values, specific values, or preferred values described herein is a preferred polymer of the invention. For a polymer of formula (15), m is an integer that is greater than or equal to 2. Prior to the polymerization illustrated in Scheme XIV, the carboxylic acid can be protected with a suitable protecting group, which can subsequently be removed, to provide the polymer of the invention.

5-Aminosalicilic acid can also be incorporated into a polymer of the invention which is a polymer of formula (23) or (24): 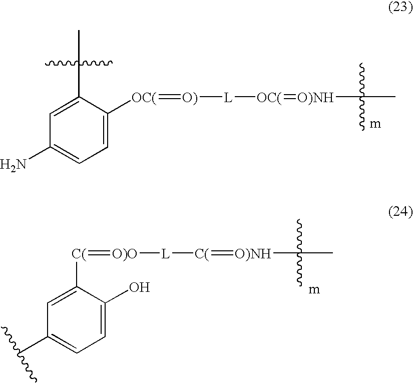
The preparation of a polymer of the invention comprising mycophenolic acid is illustrated in Scheme XV. 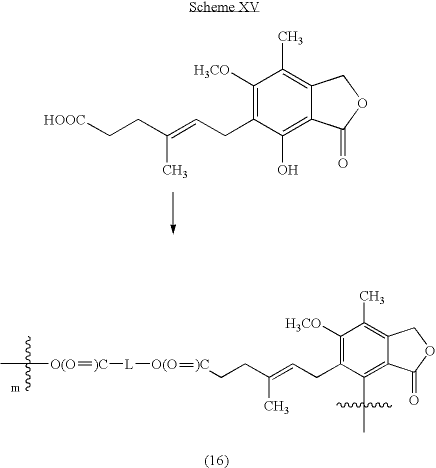
In the reaction illustrated in Scheme XV, the linking group L is preferably —(CH₂)ₓ—, and more preferably, L is —(CH₂)₈—. A polymer of formula (16) wherein L has any of the values, specific values, or preferred values described herein is a preferred polymer of the invention. For a polymer of formula (16), m is an integer that is greater than or equal to 2.

The above identified polymers, compounds and/or compositions including a biologically active agent or compound, or drug molecule of the invention can be formed into a medical implant (e.g., medical, dental, and surgical implants) or applied or coated onto a medical implant. For example, in addition to the implants described above, implants for vascular, cardiovascular, coronary, peripheral vascular, orthopedic, dental, oro-maxillary, gastrointestinal, urogenital, ophthalmic, gynecological, pulmonary, surgical, physiological, metabolic, neurological, diagnostic and therapeutic uses, may be formed from or applied or coated with the above identified polymers, compounds and/or compositions. Such implants include, but are not limited to, stents, catheters, balloons, guidewires, grafts, sutures, meshes, joint prostheses, breast prostheses, fracture management devices, drug dosing devices, pacemakers, mechanical pumps, dental implants (e.g., dental, oro-maxillary, and alveolar), defibrillators, and filters. Suitable medical implants also include, but are not limited to:
the following Boston Scientific (Boston Scientific Corporation, Natick, Mass.) products: Polaris™, NIR® Elite OTW Stent System, NIR® Elite-Monorail™ Stent System, Magic WALLSTENT® Stent System, Radius® Self Expanding Stent, NIR® Biliary Stent System, NIROYAL™ Biliary Stent System, WALLGRAFT® Endoprosthesis, WALLSTENT® Endoprosthesis, RX Plastic Biliary Stents, UroMax Ultra™ High Pressure Balloon Catheter, Passport Balloon on a Wire Catheter, Excelsior™ 1018™ Microcatheter, Spinnaker® Elite™ Flow-Directed Microcatheter, Guider Softip™ XF Guide Catheters, Sentry™ Balloon Catheters, Flexima™ APD™ Drainage Catheters with Twist Loc™ Hub, Vaxcel™ Chronic Dialysis Catheter, PASV® PICC Peripherally Inserted Central Catheters, Chilli® Cooled Ablation Catheters, and Constellation® Catheters;
the following Cordis (Cordis, a Johnson & Johnson Company, Piscataway, N.J.) products: BX Velocity™ Coronary Stents, Ninja FX™ Balloon Catheters, Raptor™ Balloon Catheters, NC Raptor™ Balloon Catheters, Predator™ Balloon Catheters, Titan Mega™ Balloon Catheters, Checkmate™ Brachytherapy Catheters, Infiniti™ Diagnostic Catheters, Cinemayre™ Diagnostic Catheters, SuperTorque Plus™ Diagnostic Catheters, and High Flow™ Diagnostic Catheters;
the following Medtronics (Medtronics, Inc., Minneapolis, Minn.) products: Aneurx Stentgraft, S7 Coronary Stents, S670 Coronary Stents, S660 Coronary Stents, BeStent 2 Coronary Stents, D1 Balloon Catheters, and D2 Balloon Catheters;
the following Avajtec Vascular (Avantec Vascular, San Jose, Calif.) products: Duraflex™ Coronary Stent System, and Apollo™ Coronary Dilatation Catheter;
the following B. Braun (B. Braun Medical Ltd., Sheffield, England) products: Coroflex™ Coronary Stent, Cystofix™ Urogenital Catheters, and Urecath™ Urogenital Catheters;
the following Cook (Cook Group Inc., Bloomington, Ind.) products: V-Flex Plus™ Coronary Stent, and CR II™ Coronary Stent;
the following Guidant (Guidant Corporation, Indianapolis, Ind.) products: Multilink Penta™ Coronary Stents, Multilink Pixel™ Coronary Stents, Multilink Ultra™ Coronary Stents, Multilink Tetra™ Coronary Stents, Multilink Tristar™ Coronary Stents, Ancure™ Stentgraft, Dynalink™ Biliary Stents, Rx Herculink™ Biliary Stents, Omnilink™ Biliary Stents, Megalink™ Biliary Stents, Rx Crosssail™ Balloon Dilatation Catheters, Rx Pauersail™ Balloon Dilatation Catheters, OTW Opensail™ Balloon Dilatation Catheters, OTW Highsail™ Balloon Dilatation Catheters, Rx Esprit™ Balloon Dilatation Catheters, Rx Viatrac™ Peripheral Catheters, and OTW Viatrac™ Peripheral Catheters;
the following Ethicon (Ethicon, a Johnson & Johnson Company, Piscataway, N.J.) products: Vicryl™ (resorbable braided coated), Pronova™, and Panacryl™;
the following USS/DG Sutures (U.S. Surgical, a division of Tyco Healthcare Group LP, Norwalk, Conn.) products: Decon II™ (coated, braided synthetic, absorbable), PolySorb™ (coated, braided synthetic, absorbable), Dexon S™ (Uncoated, braided synthetic, absorbable), Gut sutures (absorbable), Biosyn™ (synthetic monofilament, absorbable), Maxon™ (synthetic monofilament, absorbable), Surgilon™ (braided nylon, non-absorbable), Ti-Cron™ (coated, braided polyester, non-absorbable), Surgidac™ (coated, braided polyester, non-absorbable), SofSilk™ (coated, braided silk, non-absorbable), Dermalon™ (nylon monofilament, non-absorbable), Monosof™ (nylon monofilament, non-absorbable), Novafil™ (polybutester monofilament, non-absorbable), Vascufil™ (coated polybutester monofilament, non-absorbable), Surgilene™ (polypropylene monofilament, non-absorbable), Surgipro™ (polypropylene monofilament, non-absorbable), Flexon™ (stainless steel rmonofilament, non-absorbable), SURGALLOY™ needle, and SURGALLOY™ OptiVis™ needle;
the following Surgical Dynamics (Surgical Dynamics, Inc., North Haven, Conn.,) products: S*D*Sorb™ (suture anchor, Anchor Sew™ (suture anchor), S*D*Sorb E-Z Tac™ (bio-resorbable implant w/o sutures), S*D*Sorb Meniscal Stapler™ (delivers bio-absorbable repair implant), Ray Threaded Fusion Cage™ (spine), Aline™ (cervical plating system), SecureStrand™ (spinal reconstruction cable), and Spiral Radius 90D™ (spinal rod system);
the following Zimmer (Zimmer, Warsaw, Ind.) products: VerSys™ cemented stem hip system, VerSys Heritage™ Hip cemented stem hip system, VerSys™ LD/Fx cemented stem hip system, CPT™ Hip cemented stem hip system, VerSys™ Cemented Revision/Calcar cemented stem hip system, Mayor Hip porous stem hip system, VerSys™ Beaded MidCoat porous stem hip system, VerSys™ Beaded FullCoat Plus porous-stem hip system, VerSys™ Fiber Metal MidCoat porous stem hip system, and VerSys™ Fiber Metal Taper porous stem hip system, VerSys™ LD/Fx press-fit hip system, VerSys™ Cemented Revision/Calcar revision stem hip system, ZMR™ hip revision stem hip system, Trilogy™ Cup acetabular cup hip system, ZCA™ cup acetabular cup hip system, Longevity™ polyethylene hip system, Calcicoat™ coating hip system, NexGen™ Implant knee system, NexGen™ Instruments knee system, NexGen™ Revision Instruments knee system, IM™ Instruments knee system, MICRO-MILL™ 5-in-1 Instruments knee system, Multi-Reference™ 4-in-1 knee system, V-STAT™ Instruments knee system, Coonrad/Morrey™ elbow, Bigliani/Flatow™ shoulder, Cable Ready™ Cable Grip System, Collagrath™ Bone Graft Matrix, Herbert™ Bone Screw, M/DN™ Intramedullary Fixation, Mini Magna-Fx™ Screw Fixation, Magna-Fx™ Screw Fixation, Periarticular™ Plating System, Versa-Fx femoral Fixation system, Versa-Fix II™ Femoral Fixation System, and Trabecular™ Metal;
and the following Alza technologies (ALZA Corporation, Mountain View, Calif.) products: DUROS® Implant, OROS™ osmotic, D-TRANS™ transdermal, STEALTH™ liposomal, E-TRANS™ electrotransport, Macroflux™, and ALZAMER depot;
as well as those described in: Stuart, M., “Technology Strategies, Stent and Deliver,” Start-Up, Windhover's Review of Emerging Medical Ventures, pp. 34-38, June 2000); van der Giessen, Willem J., et al. “Marked Inflammatory Sequelae to implantation of Biodegradable and Nonbiodegradable Polymers in Porcine Coronary Arteries,” Circulation, Vol. 94, No. 7, pp. 1690-1697 (Oct. 1, 1996); Gunn, J. et al., “Stent coatings and local drug delivery,” European Heart Journal, 20, pp. 1693-1700 (1999);

European Patent Applications: 01301671, 00127666, 99302918, 95308988, 95306529, 95302858, 94115691, 99933575, 94922724, 97933150, 95308988, 91309923, 91906591, and 112119841;
PCT Publications: WO 00/187372, WO 00/170295, WO 00/145862, WO 00/143743, WO 00/044357, WO 00/009672, WO 99/03517, WO 99/00071, WO 98/58680, WO 98/34669, WO 98/23244, and WO 97/49434;
U.S. Application Nos. 061568, 346263, 346975, 325198, 797743, 815104, 538301, 430028, 306785, and 429459; and
U.S. Pat. Nos. 6,325,825, 6,325,790, 6,322,534, 6,315,708, 6,293,959, 6,289,568, 6,273,913, 6,270,525, 6,270,521, 6,267,783, 6,267,777, 6,264,687, 6,258,116, 6,254,612, 6,245,100, 6,241,746, 6,238,409, 6,214,036, 6,210,407, 6,210,406, 6,210,362, 6,203,507, 6,198,974, 6,190,403, 6,190,393, 6,171,277, 6,171,275, 6,165,164, 6,162,243, 6,140,127, 6,134,463, 6,126,650, 6,123,699, 6,120,476, 6,120,457, 6,102,891, 6,096,012, 6,090,104, 6,068,644, 6,066,125, 6,064,905, 6,063,111, 6,063,080, 6,039,721, 6,039,699, 6,036,670, 6,033,393, 6,033,380, 6,027,473, 6,019,778, 6,017,363, 6,001,078, 5,997,570, 5,980,553, 5,971,955, 5,968,070, 5,964,757, 5,948,489, 5,948,191, 5,944,735, 5,944,691, 5,938,682, 5,938,603, 5,928,186, 5,925,301, 5,916,158, 5,911,732, 5,908,403, 5,902,282, 5,897,536, 5,897,529, 5,897,497, 5,895,406, 5,893,885, 5,891,108, 5,891,082, 5,882,347, 5,882,335, 5,879,282, RE36,104, 5,863,285, 5,853,393, 5,853,389, 5,851,464, 5,846,246, 5,846,199, 5,843,356, 5,843,076, 5,836,952, 5,836,875, 5,833,659, 5,830,189, 5,827,278, 5,824,173, 5,823,996, 5,820,613, 5,820,594, 5,811,814, 5,810,874, 5,810,785, 5,807,391, 5,807,350, 5,807,331, 5,803,083, 5,800,399, 5,797,948, 5,797,868, 5,795,322, 5,792,415, 5,792,300, 5,785,678, 5,783,227, 5,782,817, 5,782,239, 5,779,731, 5,779,730, 5,776,140, 5,772,590, 5,769,829, 5,759,179, 5,759,172, 5,746,764, 5,741,326, 5,741,324, 5,738,667, 5,736,094, 5,736,085, 5,735,831, 5,733,400, 5,733,299, 5,728,104, 5,728,079, 5,728,068, 5,720,775, 5,716,572, 5,713,876, 5,713,851, 5,713,849, 5,711,909, 5,709,653, 5,702,410, 5,700,242, 5,693,021, 5,690,645, 5,688,249, 5,683,368, 5,681,343, 5,674,198, 5,674,197, 5,669,880, 5,662,622, 5,658,263, 5,658,262, 5,653,736, 5,645,562, 5,643,279; 5,634,902, 5,632,763, 5,632,760, 5,628,313, 5,626,604, 5,626,136, 5,624,450, 5,620,649, 5,613,979, 5,613,948, 5,611,812, 5,607,422, 5,607,406, 5,601,539, 5,599,319, 5,599,310, 5,598,844, 5,593,412, 5,591,142, 5,588,961, 5,571,073, 5,569,220, 5,569,202, 5,569,199, 5,562,632, 5,562,631, 5,549,580, 5,549,119, 5,542,938, 5,538,510, 5,538,505, 5,533,969, 5,531,690, 5,520,655, 5,514,236, 5,514,108, 5,507,731, 5,507,726, 5,505,700, 5,501,341, 5,497,785, 5,497,601, 5,490,838, 5,489,270, 5,487,729, 5,480,392, 6,325,800, 6,312,404, 6,264,624, 6,238,402, 6,174,328, 6,165,127, 6,152,910, 6,146,389, 6,136,006, 6,120,454, 6,110,192, 6,096,009, 6,083,222, 6,071,308, 6,048,356, 6,042,577, 6,033,381, 6,032,061, 6,013,055, 6,010,480, 6,007,522, 5,968,092, 5,967,984, 5,957,941, 5,957,863, 5,954,740, 5,954,693, 5,938,645, 5,931,812, 5,928,247, 5,928,208, 5,921,971, 5,921,952, 5,919,164, 5,919,145, 5,868,719, 5,865,800, 5,860,974, 5,857,998, 5,843,089, 5,842,994, 5,836,951, 5,833,688, 5,827,313, 5,827,229, 5,800,391, 5,792,105, 5,766,237, 5,766,201, 5,759,175, 5,755,722, 5,755,685, 5,746,745, 5,715,832, 5,715,825, 5,704,913, 5,702,418, 5,697,906, 5,693,086, 5,693,014, 5,685,847, 5,683,448, 5,681,274, 5,665,115, 5,656,030, 5,637,086, 5,607,394, 5,599,324, 5,599,298, 5,597,377, 5,578,018, 5,562,619, 5,545,135, 5,544,660, 5,514,112, 5,512,051, 5,501,668, 5,489,271, 6,319,287, 6,287,278, 6,221,064, 6,113,613, 5,984,903, 5,910,132, 5,800,515, 5,797,878, 5,769,786, 5,630,802, 5,492,532, 5,322,518, 5,279,563, 5,213,115, 5,156,597, 5,135,525, 5,007,902, 4,994,036, 4,981,475, 4,951,686, 4,929,243, 4,917,668, 4,871,356, 6,322,582, 6,319,445, 6,309,202, 6,293,961, 6,254,616; 6,206,677, 6,205,748, 6,178,622, 6,156,056, 6,128,816, 6,120,527, 6,105,339, 6,081,981, 6,076,659, 6,058,821, 6,045,573, 6,035,916, 6,035,751, 6,029,805, 6,024,757, 6,022,360, 6,019,768, 6,015,042, 6,001,121, 5,987,855, 5,975,876, 5,970,686, 5,956,927, 5,951,587, RE36,289, 5,924,561, 5,906,273, 5,894,921, 5,891,166, 5,887,706, 5,871,502, 5,871,490, 5,855,156, 5,853,423, 5,843,574, 5,843,087, 5,833,055, 5,814,069, 5,813,303, 5,792,181, 5,788,063, 5,788,062, 5,776,150, 5,749,898, 5,732,816, 5,728,135, 5,709,067, 5,704,469, 5,695,138, 5,692,602, 5,683,416, 5,681,351, 5,675,961, 5,669,935, 5,667,155, 5,655,652, 5,628,395, 5,623,810, 5,601,115, 5,571,469, 5,555,976, 5,545,180, 5,529,175, 5,500,991, 5,495,420, 5,491,955, 5,491,954, 5,487,216, 5,487,212, 5,486,197, 5,485,668, 5,477,609, 5,473,810, 5,409,499, 5,364,410, 5,358,624, 5,344,005, 5,341,922, 5,306,280, 5,284,240, 5,271,495, 5,254,126, 5,242,458, 5,236,083, 5,234,449, 5,230,424, 5,226,535, 5,224,948, 5,213,210, 5,199,561, 5,188,636, 5,179,818, 5,178,629, 5,171,251, 5,165,217, 5,160,339, 5,147,383, 5,102,420, 5,100,433, 5,099,994, 5,089,013, 5,089,012, 5,080,667, 5,056,658, 5,052,551, 5,007,922, 4,994,074, 4,967,902, 4,961,498, 4,896,767, 4,572,363, 4,555,016, 4,549,649, 4,533,041, 4,491,218, 4,483,437, 4,424,898, 4,412,614, D260,955, 4,253,563, 4,249,656, 4,127,133, D245,069, 3,972,418, 3,963,031, 3,951,261, 3,949,756, 3,943,933, 3,942,532, 3,939,969, 6,270,518, 6,213,940, 6,203,564, 6,191,236, 6,138,440, 6,135,385, 6,074,409, 6,053,086, 6,016,905, 6,015,427, 6,011,121, 5,988,367, 5,961,538, 5,954,748, 5,948,001, 5,948,000, 5,944,739, 5,944,724, 5,939,191, 5,925,065, 5,910,148, 5,906,624, 5,904,704, 5,904,692, 5,903,966, 5,891,247, 5,891,167, 5,889,075, 5,865,836, 5,860,517, 5,851,219, 5,814,051, 5,810,852, 5,800,447, 5,782,864, 5,755,729, 5,746,311, 5,741,278, 5,725,557, 5,722,991, 5,709,694, 5,709,692, 5,707,391, 5,701,664, 5,695,879, 5,683,418, 5,669,490, 5,667,528, 5,662,682, 5,662,663, 5,649,962, 5,645,553, 5,643,628, 5,639,506, 5,615,766, 5,608,962, 5,584,860, 5,584,857, 5,573,542, 5,569,302, 5,568,746, 5,566,822, 5,566,821, 5,562,685, 5,560,477, 5,554,171, 5,549,907, 5,540,717, 5,531,763, 5,527,323, 5,520,702, 5,520,084, 5,514,159, 5,507,798, 5,507,777, 5,503,266, 5,494,620, 5,480,411, 5,480,403, 5,462,558, 5,462,543, 5,460,263, 5,456,697, 5,456,696, 5,442,896, 5,435,438, 5,425,746, 5,425,445, 5,423,859, 5,417,036, 5,411,523, 5,405,358, 5,403,345, 5,403,331, 5,394,971, 5,391,176, 5,386,908, 5,383,905, 5,383,902, 5,383,387, 5,376,101, D353,672, 5,368,599, D353,002, 5,359,831, 5,358,511, 5,354,298, 5,353,922, 5,350,373, 5,349,044, 5,335,783, 5,335,775, 5,330,442, 5,325,975, 5,318,577, 5,318,575, 5,314,433, 5,312,437, 5,310,348, 5,306,290, 5,306,289, 5,306,288, 5,294,389, 5,282,832, 5,282,533, 5,280,674, 5,279,783, 5,275,618, 5,269,807, 5,261,886, 5,261,210, 5,259,846, 5,259,845, 5,249,672, 5,246,104, 5,226,912, 5,225,485, 5,217,772, 5,217,486, 5,217,485, 5,207,679, D334,860, 5,197,597, 5,192,303, D333,401, D333,400, 5,181,923, 5,178,277, 5,174,087, 5,168,619, 5,163,946, 5,156,615, 5,154,283, 5,139,514, 5,133,738, 5,133,723, 5,131,534, 5,131,131, 5,129,511, 5,123,911, 5,121,836, 5,116,358, 5,102,418, 5,099,676, 5,092,455, 5,089,011, 5,089,010, 5,087,263, 5,084,063, 5,084,058, 5,078,730, 5,067,959, 5,059,213, 5,059,212, 5,051,107, 5,046,513, 5,046,350, 5,037,429, 5,024,322, 5,019,093, 5,002,550, 4,984,941, 4,968,315, 4,946,468, 4,932,963, 4,899,743, and 4,898,156;
   which are each hereby incorporated by reference in their entirety.

In addition to those set forth above, examples of suitable classes of a biologically active agent or compound or drug molecule for inclusion in or addition to a biocompatible and biodegradable polymer or composition include, but are not limited to, an antineoplastic agent or anti-metabolite agent (e.g., cladribine, camptothecin, irinotecan, topotecan, paclitaxel, methotrexate, vincristine, actinomycin-D), an immunosuppressant (e.g., rapamycin, thalidomide), an anti-thrombogenic or anticoagulant agent (e.g., fibrin, heparin binding growth factor, sodium heparin, low molecular weight heparin, hirudin, argatroban, vapiprost, D-phe-pro-arg-chloromethylketone, dipyridamole, glycoprotein IIb/IIIa inhibitors, platelet membrane receptor antibody, recombinate hirudin, thrombin inhibitor, dextran, activated protein C), an anti-inflammatory agent (e.g., salicylate (e.g., salicylic acid, aspirin, 4-aminosalicylate, 5-aminosalicylate), ketoprofen, steroids (e.g., dexamethasone, glucocorticoids, methylprednisolone, prednasone, methylprednasone, hydrocortisone), naproxyn, ibuprofen, flurbuprofin), an anti-fungal, an anti-bacterial, an anti-viral, an anti-infective or anti-biotic agent (e.g., amoxicillin, penicillin, ciprofloxacin), a prostaglandin or prostaglandin inhibitor, an angiotensin converting enzyme inhibitor, a calcium channel blocker, oils (e.g., fish oil, omega 3-fatty acid), a histamine antagonist, a HMG-CoA reductase inhibitor, a monoclonal antibody, a serotonin blocker, a phophodiesterase inhibitor, an alpha-interferon, genetically engineered epithelial cells and combinations thereof, a quinazolinone derivative, a nucleic acid encoding an endothelial cell mitogen such as vascular endothelial growth factor (VEGF), vitamin (e.g., alpha-tocopherol, vitamin D), a growth factor (e.g., fibroblast growth factor antagonists, platelet derived growth factors and antagonists, a bone growth factor (e.g., osteopontin, bisphosphonates (e.g., risedronate, etidonate, alendronate) and estrogen receptor modulators (e.g., raloxifene)), an antioxidant, an endothelin receptor antagonist, an angiopeptin, DNA and DNAzymes, a tyrosine kinase inhibitor ST638, a polynitrosylated albumin NO donor, a natural, semi-synthetic or synthetic hormone (e.g., follicle stimulating hormone (F.S.H.) and lutenizing hormone (L.H.)), and an anti-sense to targets effected by drugs listed above, and mixtures of one or more of the above biologically active agents or compounds, or drug molecules.

The term “formed into” includes within its meaning that a polymer, compound and/or composition of the invention can be physically configured into various shapes, geometries, structures and configurations including, but not limited to, a film, fiber, rod, coil, corkscrew, hook, cone, pellet, tablet, tube (smooth or fluted), disc, membrane, microparticle, “biobullet” (i.e., bullet shaped), seed (i.e., bullet shaped or targeted seeds), as well as those described in the above identified products, patents and articles, including in some cases forming medical implants that have the same, similar or completely different functional characteristics compared to those functional characteristics of the medical implants described in the above identified products, patents and articles. The above-mentioned shapes, geometries, structures and configurations may contain additional features that will further enhance the desired application or use. For example, a polymer, compound and/or composition of the invention in the form of a rod, coil, or cone may have barbs that spring out upon insertion from a needle or canula or when warmed to body temperature to reduce movement and/or expulsion.

The shape, geometry, structure or configuration of a medical implant of the invention will vary depending upon the use of the implant. For example, for treatment of a spinal cord injury or concussion to the brain, a polymer, compound and/or composition of the invention can be formed into a medical implant in the shape of a disc for placement under the dura or dura mater. In another example, a polymer, compound and/or composition of the invention can be formed into a medical implant in the shape of a membrane or tube for use in the treatment of injury or damage to the peripheral nervous system or a block of solid or foamed composition containing pathways drilled or otherwise formed to encourage nerve growth or bone growth. In another example, in the treatment of cancer, a polymer, compound and/or composition of the invention can be formed into a medical implant in the shape of a pellet, microsphere, rod; membrane, disc, bullet, hook rod or cone, with or without barbs, for insertion in a tumor excision site or for insertion within a tumor. In the above instances, bioerosion of the medical implant would yield or generate an active agent.

The invention also contemplates that the shape, geometry, structure or configuration of a medical implant of the invention can change depending-on the mode of delivery or administration and can enhance the therapeutic effect of the medical implant. For example, a medical implant of the invention nay be in the form of a linear rod when inserted in needles and stored but may become coil-like or form a multiplicity of coils or corkscrew shapes as the medical implant is pushed out of the needle by a trochar. As a result of the change of the shape, geometry, structure or configuration of the medical implant, expulsion from the tumor or tumor excision site by hydraulic pressures or body movements can be prevented and as much mass of active ingredient can be delivered to a small region with as small a diameter needle as possible.

The mode of delivery or administration of a medical implant of the invention may vary depending upon the desired application and include those known in the art as well as those set forth herein.

A polymer, compound and/or composition of the invention can be formed into a medical implant by any means known in the art including, but not limited to, molding (e.g., compression or blow molding) and extrusion. The medical implant may be formed from one or more of the same or different polymer, compound and/or composition of the invention.

A polymer, compound and/or composition of the invention can also be applied or coated onto a medical implant by any means known in the art including, but not limited to, solvent methods such as, for example, dipping and spray-drying, and non-solvent methods such as chemical vapor deposition, extrusion coating or dipping in molten polymer, compound and/or composition of the invention. The method of preparation may vary depending on the polymer, compound and composition and/or the medical implant. The medical implant can be formed from or coated with one or more layers of the same or different polymer, compound and/or composition of the invention.

In another example, a polymer, compound and/or composition of the invention can be coated onto a medical implant in the shape of a membrane or tube for use in the treatment of injury or damage to the peripheral nervous system or a block of solid or foamed composition containing pathways drilled or otherwise formed to encouraged nerve growth or bone growth. In the above instances, bioerosion of the disc, membrane, tube or block would yield or generate an active agent included within the polymer or composition.

The thickness of the polymer, compound and/or composition as either the medical implant itself or as applied or coated onto a medical implant will vary depending upon one or more factors such as the physical and/or chemical characteristics of the polymer, compound and/or composition, the medical implant and/or the application or use.

For example, a coronary artery stent may be formed from or applied or coated with a polymer, compound and/or composition of the invention to a thickness of about ≦30-50 μm while a vascular stent may be applied or coated with a polymer, compound and/or composition of the invention to a thickness of about ≦100 μm and a drug delivery device may be applied or coated with a polymer, compound and/or composition of the invention to a thickness of about ≦2 mm. In another example, round films/membranes for buccal (sublingual) administration (e.g., placement in lining of cheek, under the tongue) will have diameters of up to about 10 mm (2 cm) and a thickness of about 0.5-2.0 mm.

Further the polymers, compounds and/or compositions of the invention can be formed into micronized particles or microparticles (e.g., microspheres and/or microcapsules). Microparticles of a polymer, compound and/or composition of the invention may be prepared by any means known in the art and may include one or more of the same or different polymer, compound and/or composition of the invention. For example, the microparticles can be prepared using an oil-in-water emulsion method whereby a polymer of the invention is dissolved in an organic solvent. The polymer solution is then added to a stirring solution of water and PVA (polyvinyl alcohol, which stablilizes the microparticle) resulting in the percipitation of the desired microparticles. Optionally, a homogenizer could be used. The solution is then allowed to settle, the solvent is decanted off the solution and the microparticles are then dried.

In another oil-in-water emulsion method, the polymer solution is added to a solution of water and a surfactant such as PVA, which is stirred rapidly at high shear rates with, for example, a homogenizer or dispersator. After the addition of the polymer solution, the solvent is allowed to evaporate while stirring is continued. The resulting microparticles are recovered by decantation, filtration or centrifugation and dried.

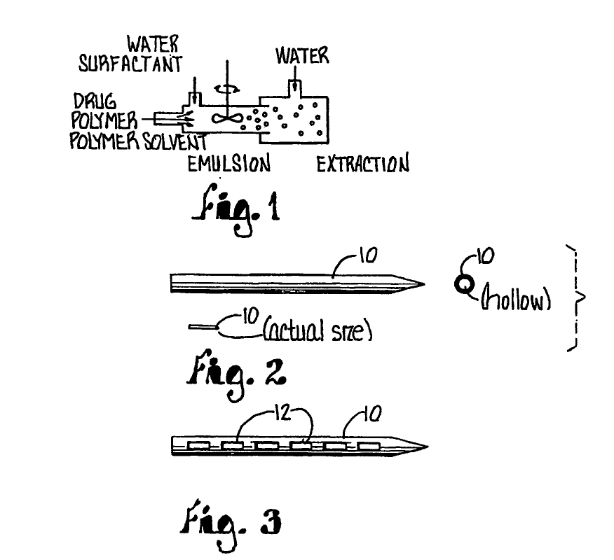

A microparticle of the invention can also be prepared by Southern Research's (Southern Research Institute, Birmingham, Ala.) continuous microencapsulation process as set forth in U.S. Pat. No. 5,407,609, which is incorporated herein by reference in its entirety, and is described in FIG. 1, attached hereto.

According to Southern Research's continuous microencapsulation process described in FIG. 1, proteins, peptides, small molecules, water-soluble drugs, hydrophobic drugs, and drugs encapsulated in lactide/glycolide polymers can be microencapsulated to sizes of about 1-250 μm preferably <100 μm, more preferably, <10 μm with minimal exposure to polymer solvent, high encapsulation efficiency and good yields. As shown in FIG. 1, a drug, polymer and polymer solvent dispersion is added to a mechanically agitated water/surfactant mixture to form an emulsion of microdroplets, which is then extracted with water to remove solvent and produce hardened microcapsules or microspheres for collection by centrifugation, filtration or the like.

The microparticles of the invention may be formed into various shapes and geometries (e.g., spheres, and regular or irregular spheroid shapes) as well as incorporated into various formulations or compositions (e.g., gelatin capsule, liquid formulation, spray dry formulations, formulations for use with dry powder or aerosol inhalers, compressed tablet, topical gels, topical ointments, topical powder).

As would be understood by one of skill in the art, the desired size of a microparticle of the invention will depend on the desired application and mode of delivery. Modes of administration or delivery of a microparticle of the invention include those set forth herein, including orally, by inhalation and topically. The present invention contemplates the administration of a microparticle of the invention which upon degradation or bioerosion yields a smaller particle and/or active agent for the effective treatment of a targetted organ. The present invention also contemplates administration of one or more of the same or different microparticles of the invention having either all the same size or a mixture of two or more different sizes. By varying the size of the microparticle, the rate of bioerosion and/or the rate of generation of active drug and/or the location of active drug generation can be controlled. As a result, timed (e.g., delayed and/or sustained) generation of active drug can be achieved.

For example, treatment of the inflamed wall of the colon (e.g., the treatment of inflammatory bowel disease, infections, and the like) may be achieved by oral administration of a microparticle of the invention containing as the active agent an anti-inflammatory drug. Such a microparticle of about 1-10 μm in size may be administered such that upon reaching the ileum region of the small intestine, the microparticle is about 0.1-1.0 μm in size, and about 0.01-0.1 μm size upon reaching the colon. See for example, A. Lamprecht et al., Abstracts/Journal of Controlled Release, Vol. 72, pp. 235-237 (2001). Once in the intestine, the microparticle can be physically entrapped by the villi and/or microvilli of the intestinal wall and/or by the mucous lining of the intestinal wall, thereby retarding expulsion, and prolonging gastrointestinal residence time and enabling timed sustained generation of the active agent in the proximity of the intestinal wall upon bioerosion of the polymer.

Similarly, about 0.1-100 μm, preferably about 0.1-10 μm, more preferably about 0.1-1 μm, microparticle of the invention may be administered orally such that blood levels of the microparticle enable perfusion of the active agent into the surrounding tissue upon bioerosion. In yet another example, oral administration of a microparticle of the invention of about ≦0.6 μm, preferably about ≦0.3 μm, more preferably about 0.1 μm, may be used to deliver an active drug through the intestine and eventually to the liver via the lymph system. See for example, P. Jani et al., Pharm. Pharmacol., Vo. 42, pp. 821-826 (1990); M. Desai et al., Pharmaceutical Research, Vol. 13, No. 12, pp. 1838-1845 (1996)

A microparticle of the invention of about ≦10 μm may be applied topically or ocularly.

For skin penetration, about 1-70 μm microparticle of the invention may be used. In one preferred embodiment, about 10-70 μm microparticle of the invention is used for skin penetration. In another preferred embodiment, ≦10 μm microparticle of the invention is used to create a product that feels smooth when applied to human skin. In another preferred embodiment, about 1-3 μm microparticle of the invention is used for skin penetration. However, various microparticle sizes may be used, as exemplified in PowderJect's Smart Particle™ (PowderJect Pharmaceuticals, England, U.K., including those described in U.S. Pat. Nos. 6,328,714, 6,053,889 and 6,013,050) in tissue (e.g., skin, mucosa) penetration applications which appear to rely more on shape and strength of the microparticle rather than size.

A microparticle of the invention may also be used in an inhaled delivery (e.g., direct inhalation at a certain velocity, or by aerosol spray) to the lungs, including deep lungs, or pulmonary region. For example, a microparticle of the invention of about 0.5-10 μm, preferably about 1-5 μm, more preferably about 1-3 μm even more preferably about 1-2 μm may be formulated into an aerosol. For direct inhalation, about 0.5-6 μm, more preferably about 1-3 μm, microparticle may be used. See for example, ARADIGM's (Aradigm Corporation, Hayward, Calif.) AERx® System as well as those described in U.S. Pat. Nos. 6,263,872, 6,131,570, 6,012,450, 5,957,124, 5,934,272, 5,910,301, 5,735,263, 5,694,919, 5,522,385, 5,509,404, and 5,507,277, and MicroDose's (MicroDose Technologies Inc., Monmouth Junction, N.J.) MicroDose DPI Inhaler as well as those described in U.S. Pat. Nos. 6,152,130, 6,142,146, 6,026,809, and 5,960,609.

A microparticle of the invention of about ≦10 μm may be used for intraarticular injections in the treatment of, for example, arthritis.

A microparticle of the invention of about 0.1-100 μm, preferably about 0.1-10 μm, more preferably about 0.1-1 μm, may be admixed with a suppository (e.g., glycerin suppository).

A polymer, compound and/or composition of the invention may also be formed into pellets, “biobullets” (i.e., bullet shaped) or seeds (e.g., bullet-shaped seeds) for inclusion in an implantable and/or injectable bioerodable, hollow carrier 12 (e.g., barrel, bullet, capsule, syringe or needle) as exemplified in FIGS. 2 and 3. Both animal and human applications are contemplated. FIG. 2 illustrates several hollow needle-type carriers 12 for use in the invention. In one embodiment, hollow carriers 12 have a diameter ranging from about 0.5-10 mm.

FIG. 3 illustrates placement of pellets, “biobullets,” or seeds 10 of the invention inside the hollow cavity or chamber of a bioerodable needle-type carrier. According to the invention, one or more of the same or different pellet, “biobullet,” or seed 10 of the invention may be placed inside the hollow carrier 12 or delivery device. The pellet, “biobullet” or seed 10 may be any size that will enable placement inside the hollow carrier 12.

According to the invention, upon bioerosion of the pellet, “biobullet,” or seed 10, an active agent is generated.

The invention also contemplates that the hollow carrier 12 may also be formed from a polymer, compound and/or composition of the invention such that upon bioerosion of the hollow carrier 12, an active agent may be released and/or its contents (e.g., pellets, “biobullets” or seeds of the invention) may be released.

In one preferred embodiment, pellets, “biobullets,” or seeds 10 are made from a polymer of the invention containing salicylic acid admixed with follicle stimulating hormone (F.S.H.) and/or lutenizing hormone (L.H.) which are then placed in the hollow cavity or chamber of a bioerodable hollow carrier 12 or as part of a depot formulation (e.g., Lupron Depot®) for a timed release delivery of the hormones up to about 96 hours in order to stimulate ovulation.

According to the invention, a pellet, “biobullet” or seed 10 of the invention and/or one or more hollow carriers 12 containing a pellet, “biobullet,” or seed 10 of the invention may be placed in a delivery device (e.g., injector, gas-driven applicator). The delivery device may be further equipped with an axially slidable sleeve (e.g., plunger), protrusions to prevent movement of the delivery device upon application (e.g., chamfered protrusions), and handgrips. Examples of suitable carriers and/or delivery devices include, but are not limited to, those described in U.S. Pat. Nos. 6,001,385, 5,989,214, 5,549,560; WO 96/13300, WO 96/09070, WO 93/23110, and EP 068053, each of which is herein incorporated by reference in its entirety.

For example, U.S. Pat. No. 5,989,214 and WO 96/13300 describe an apparatus for injecting the body of humans or animals with a pharmaceutical preparation, wherein the preparation is arranged in a rigid carrier, wherein the apparatus includes: a chamber into which the carrier can be transported; and a channel connecting onto the chamber for transporting the carrier into the body including fixation means for fixing the end of the channel relative to the skin of the body for injecting in order to prevent a movement of the channel in the direction perpendicularly of the axis of the barrel and where according to one embodiment the fixation means are formed by chamfered protrusions formed on the part adapted for contact with the skin of the body and extending substantially in the direction of the axis of the channel. U.S. Pat. No. 5,549,560, WO 93/23110, and BP 068053 describe a device for injecting humans and animals with a pharmaceutical preparation, wherein the preparation is held in a rigid carrier and the carrier is carried through the skin into the body by means of gas pressure, and wherein during carrying of a rigid carrier into the body by means of gas pressure the device with which the carrier is carried into the body is held against the body. U.S. Pat. No. 5,549,560, WO 93/23110, and EP 068053 also describe a device for injecting animals or humans with a pharmaceutical preparation, wherein a chamber is present in which a carrier containing the pharmaceutical preparation can be placed, a barrel connecting onto this chamber and means for carrying the carrier by means of gas pressure through the barrel into the body for injecting, wherein means are present for blocking the use of the device when it is not pressed against a body. U.S. Pat. No. 6,001,385 and WO 96/09070 describe “bullets” that are at least partly manufactured from substantially fully destructurized starch, particularly implants, suitable as vehicles for introducing active agents into the human or animal body in a transdermal manner.

The range of therapeutically effective dosages, that is, the dosage levels necessary to achieve the desired result, of a microparticle of the invention will be influenced by the route of administration, the therapeutic objectives, and the condition of the patient. As such, a microparticle of the invention may be administered as a single daily dose, several times daily, every other day, weekly, etc. depending on the dosage requirements. Individual determinations will need to be made to identify the optimal dosage required.

A polymer, compound and/or composition of the invention may be combined or admixed with other ingredients prior to or while being formed into or coated onto a medical implant or microparticle or into a particular coating for a medical implant. Examples of suitable additives include, but are not limited to, stabilizers, mechanical stabilizers, plasticizers, hardeners, emulsifiers, other polymers including other biocompatible and biodegradable polymers (e.g., biocompatible and biodegradable polyanhydrides as set forth in U.S. application Ser. No. 09/917,231 and PCT Application No. US/01/23740, biocompatible and biodegradable polyazo compounds as set forth in U.S. application Ser. No. 09/917,595 and PCT Application No. US/01/23748, biocompatible and biodegradable polyesters, polythioesters, and polyamides as set forth in U.S. application Ser. No. 09/917,194 and PCT Application No. US/01123747, each of which is incorporated by reference in its entirety), radioopaque and/or radioisotopic materials (e.g., boron, iodine, etc.), suppositories, and other diagnostic or therapeutic agents or drugs.

An added ingredient may enhance stability of the polymer, compound and/or composition itself, the medical implant itself and/or may enhance the diagnostic or therapeutic effect and/or may enhance or enable diagnostic activity. For example, if the added ingredient is a diagnostic or therapeutic agent or drug, bioerosion of the polymer would not only generate the active agent but would also release the diagnostic or therapeutic agent. In another example, by adding a radioopaque material visualization of both the targeted area (e.g., tumor site, tumor) and the medical implant (e.g., catheter) would be enabled during and/or after (e.g., angioplasty, dental applications, joint injections, etc) insertion of the medical implant. In another example, the radioopaque material may also be used to control and/or enhance bioerosion of the medical implant and thereby control and/or enhance generation of the active agent by the generation of heat resulting from neutron capture.

An added ingredient may also enhance the overall mechanical stability of the medical implant (e.g., carbon fibers). The type of additive used would vary and depend upon the desired property and application.

Activity

The ability of a polymer of the invention to produce a given therapeutic effect can be determined using in vitro and in vivo pharmacological models which are well known to the art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. (canceled)

2. (canceled)

3. A method of treating arthritis in a mammal in need thereof, comprising injecting into the area of the arthritis an effective amount of a polymer, wherein the polymer comprises a backbone which incorporates linkages selected from the group consisting of ester, thioester and amide and the backbone incorporates at least one group which will yield a biologically-active anti-inflammatory upon the hydrolysis of the polymer in the mammal.

4. The method of claim 3 wherein the polymer is selected from the group consisting of a repeating unit of Formula 1 and Formula 2
—R¹-A-L-A (1)
—R²-A-L-A-R³-A-L-A- (2)
wherein each R¹, R², and R³, independently from one another, comprises at least one anti-inflammatory agent having two reactive functional groups; each A, independently from one another, comprises an ester, thioester or amide moiety and L comprises a linking moiety whose reactive functional group forms a part of A and wherein a reactive functional group of R¹, R² or R³ forms a part of A, which polymer yields a biologically-active anti-inflammatory agent upon the hydrolysis of the polymer.
